(19) United States
(12) Patent Application Publication
(10) Pub. No.: US 2006/0088479 A1
(43) Pub. Date: Apr. 27, 2006

(54) Particle formulation and its preparation

(76) Inventor: Nicola Kim Whitfield, Nottingham (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/546,844

(22) PCT Filed: Feb. 27, 2004
(86) PCT No.: PCT/GB04/00786

§ 371 Date: Nov. 16, 2005

(30) Foreign Application Priority Data
Feb. 27, 2003 (GB) 0304540.8

(51) Int. Cl. A61K 9/14; A61L 9/04

(52) U.S. Cl. 424/46

(57) ABSTRACT

Powder compositions are prepared which compose microparticles having a bioactive agent dispersed therein. The compositions are prepared by spray drying a solution or suspension of the bioactive and collecting the resulting microparticles using a separator.

### FIELD OF THE INVENTION

This invention relates to a particle formulation and its preparation. In particular, it relates to the formation of particle assemblies, to improve powder flow.

### BACKGROUND OF THE INVENTION

Fine powders in the range of 1-5 μm Mass Median Aerodynamic diameter (MMAD) are characteristically cohesive, with poor flow properties. Small changes in moisture, high surface area and small size, as well as surface charge, morphology and density all influence the flowability of such powders. When such cohesive powders agglomerate, they can often be difficult to separate, e.g., when the powder is dispersed using a dry powder inhalation device.

Several methods are available to collect powders. Typically, spray-dried powders are collected using cyclone technology, which may be combined with some form of metal grid screen. Bag filters are known and available, e.g. from Niro, Fairey, etc.

Spray-dried powders collected into a vessel using a cyclone arrangement undergo partial classification, losing many of the smaller particles in the waste exhaust, in particular, those significantly less than 1 μm in size. Yields of such desired small particles can be as high as 70%, though are typically in the order of 50-60%. The resultant collected powders show excellent dispersion characteristics and are effectively deaggregated using new generation dry powder inhalation devices. However, these powders often exhibit extremely poor flow characteristics, and hence become difficult to fill into suitable unit doses for use in DPIs, e.g. blister wells, capsules, etc. particularly at small quantities (e.g. <5 mg).

A problem with many collection systems is that binding can occur very rapidly, reducing air flow, as well as exposing any “dried” product to unnecessary extended periods in a potentially hot and moist air stream. The powders recovered tend to be severely agglomerated and require aggressive milling processes to produce discrete particles.

### SUMMARY OF THE INVENTION

The present invention is based on the surprising finding that particular size ratios of microparticles can be obtained which exhibit good flow properties. These particle assemblies (PAs) can be easily broken down into their primary particles upon device actuation and subsequent administration to a patient. The compositions therefore have beneficial characteristics for the delivery of bioactive agents to a patient via the pulmonary route.

According to a first aspect of the invention, a composition comprises microparticles which comprise a bioactive agent, wherein
(1) at least 40% of the microparticles are from 1 μm to 5 μm MMAD;
(2) at least 40% of the microparticles are less than 1 μm; and
(3) at least 2% of the microparticles are less than 0.5 μm.

According to a second aspect of the invention, a device for the pulmonary delivery of a bioactive agent comprises a composition as defined above.

According to a third aspect of the invention, a method for the preparation of a composition as defined above comprises spray drying a solution or suspension of the agent and optional additives, in a spray dryer under conditions suitable to provide microparticles of less than 5 μm MMAD, the apparatus having a separator to remove the spray dried powders from the gas stream, and collecting those microparticles retained by the separator.

The composition of the invention can be readily filled into a container, e.g. a blister pack. Such a composition is particularly intended for use in a dry powder inhaler device. By means of the invention, the fine particle fraction generated by the device is not reduced when particle assemblies (PAs) formed using the method are employed.

### DESCRIPTION OF THE DRAWINGS

The invention is described with reference to the accompanying drawings, wherein:

FIG. 1 is a graphic illustration of the uniformity of filling a blister pack using compositions of the invention;

FIGS. 2-4 are SEM images of microparticles prepared either using a cyclone separator or a filter.

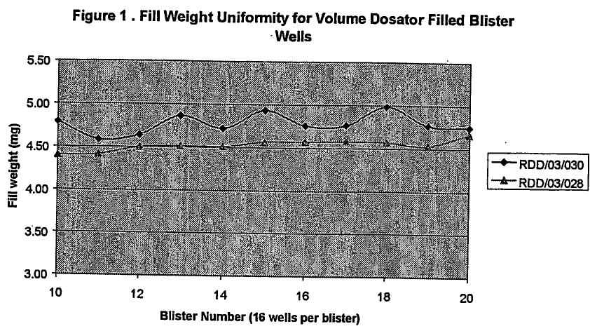

### DESCRIPTION OF THE INVENTION

The present invention makes use of conventional spray drying equipment to produce compositions having beneficial ratios of different sized microparticles.

The microparticles may be formulated with any suitable bioactive agent. The term “bioactive” is intended to include any pharmacologically active agent, useful for treatment or prophylaxis. Suitable bioactive agents include, but are not limited to, peptides or proteins, hormones, analgesics, anti-migraine agents, anti-coagulant agents, narcotic agents, antagonists, anti-anginal agents, anti-asthmatic agents and cardiovascular drugs. Preferred bioactive agents include insulin, erythropoietin (EPO), interferon (α, β or γ), somatrotopin, somatostatin, tissue plasminogen activator (TPA), factor VIII and interleukin. Immunogens may also be used in the prophylaxis of any bacterial or viral disease.

In one embodiment, the microparticles of the invention are formulated with a carbohydrate. The carbohydrate matrix within which the bioactive is dispersed may be crystalline or amorphous. In a preferred embodiment, the microparticles of the invention are amorphous with a glass transition temperature above 20° C., as measured by Differential Scanning calorimetry. Suitable carbohydrates include any monosaccharide, disaccharide, oligosaccharide or their corresponding sugar alcohols. Preferred carbohydrates include trehalose, sucrose and raffinose.

In the absence of carbohydrate, the microparticles may also be amorphous or crystalline.

The composition of the invention will comprise different ratios of different sized microparticles. Without being bound by theory, it seems that the submicron particles are able to form reversible agglomerates or particle assemblies comprising the larger particles during the spray-drying process or it may be that these sub-micron particles reduce the Van der Waals cohesive forces between the larger particles, thus improving the flow characteristics of the composite. Alternatively, again not wishing to be bound by theory, the particles may be acquiring frictional charge due to their constant vibration against a dissimilar surface, e.g., a sintered metal or PTFE filter membrane (used as the separator). Indeed, surface amorphicity has been shown to have a significant role in the charging properties of aerosols. For example, the frictional charge between lactose and polypropylene increases with increasing amorphicity.

The composition will comprise typically:
(1) at least 40% microparticles from 1 μm to 5 μm MMAD;

(2) at least 40% microparticles of less than 1 μm; and

(3) at least 2% microparticles of less than 0.5 μm.

In a preferred embodiment, at least 50% of the microparticles are less than 1 μm and at least 5% of the microparticles are less than 0.5 μm. In a more preferred embodiment, at least 20% of the microparticles are less than 0.75 μm. In a more preferred embodiment, all of the microparticles in the composition are less than 5 μm, preferably less than 4 μm.

The microparticles in the composition form loose agglomerates and have a fine particle dose of greater than 30% less than 3.3 μm of detected bioactive.

The MMAD may be measured using an Aerosizer (TSI Instruments) as will be appreciated by the skilled person.

The compositions of the invention may be prepared using conventional spray drying apparatus, for example a mini spray dryer or a conventional scale spray dryer (e.g. a Niro Mobile Minor).

An important aspect of the present invention is the use of a separator to remove the spray dried powders from the gas stream. Suitable separators include bag collectors, cloth filters, bag filters, sintered metal filters, etc which are available commercially from Fairey, Niro, Ohkawara, etc. The filter may be made from any suitable material, such as metal, polyester or polytetrafluoroethylene. The microparticles of the composition are retained in or on the separator, and are not collected from a cyclone, as in conventional spray dry techniques.

When formulating the microparticles with a carbohydrate, it is preferable to employ a two fluid nozzle. Other suitable atomisers include pneumatic, rotary, piezoelectric, etc. Multiple orifice configurations are also suitable for use in this invention. In a further embodiment, a three-fluid atomiser may be employed. This allows the bioactive agent and the additives to be delivered to the dryer from separate feedstocks. Each feed may be directed to separate atomisers within the spray dryer. This is beneficial when it is desirable to dissolve or suspend the bioactive agent in one solvent and the additive in a different solvent. For example, a dilute solution or suspension of a suitable hydrophobic flow enhancer, e.g. leucine, trileucine, magnesium stearate, may be atomised separately from the active, thereby producing an agglomerate comprising a submicron population of flow enhancer. Alternatively, a highly charged material, anionic (e.g. hyaluronic acid) or cationic (e.g. polyglutamic acid), may be substituted for the flow enhancer.

The conditions for the spray drying process are selected to provide microparticles of less than 5 μm MMAD in diameter. It is preferable to carry out spray drying with an outlet temperature of at least 70° C. The inlet temperature will be selected to achieve the specified outlet temperature, based on the size and type of spray dryer being used: The inlet temperature may be 200° C. or more for a large scale dryer but at least 100° C. for a small scale dryer. The feed-rate will also vary depending on the spray drier used. It is preferable for a mini spray dryer to operate with a feed-rate between 1-5 g/min while a large scale dryer (e.g. Niro Mobile Minor) is operated at a feed-rate from 5-16 g/min, preferably at 16 g/min.

The atomisation pressure will also be selected to achieve microparticles of less than 5 μm MMAD. An atomisation pressure of at least 6 bar with an air flow of at least 10 l/sec for a large scale dryer is suitable.

It will be appreciated that the microparticles are formulated in physiologically effective amounts. That is, when delivered from a unit dosage form, there should be a sufficient amount of the bioactive agent delivered to the desired location to achieve the desired response. Typically, the yield of microparticles will be at least 60% of the solids content present in the solution or suspension prior to spray drying. Preferably the microparticles comprise at least 70%, more preferably 80% and most preferably at least 90% of the bioactive agent present in the original solution or suspension. If an additive is to be used, for example a carbohydrate, the ratio of bioactive to additive in the original solution or suspension will preferably be at least 50:50, more preferably 60:40, and most preferably at least 70:30 w/w.

The microparticle compositions are suitable for pulmonary delivery to a patient. Devices suitable for delivery of the compositions are known, and will be apparent to the skilled person. The preferred delivery system is a passive dry powder inhaler (DPI), which relies entirely on the patient's inspiratory efforts to introduce the particles in a dry powder form into the lungs. However, alternative delivery devices may also be used. For example, active inhalers employing a mechanism or piezoelectric device for delivering the powder to the patient may be used. The microparticles may be formulated for delivery using a metered dose inhaler (MDI), which usually requires a high vapour pressure propellant to force the particles from the device. Although pulmonary delivery is preferred, the compositions of the invention may be used via any other suitable route.

The following Examples demonstrate the preparation of compositions according to the invention.

### EXAMPLE 1

TABLE 1

| | Insulin | 0.05M | 1.0M | Trehalose | Water |
|---|---|---|---|---|---|
| Batch | (g) | HCL (ml) | NaOH (ml) | (g) | (ml) |
| RDD/03/008 | 10 | 125 | 9.3 | 0 | 97.1 |
| RDD/03/027 | 20 | 250 | 18.6 | 0 | 731.4 |
| RDD/03/030 | 4 | 50 | 3.72 | 16 | 946.3 |
| RDD/03/028 | 4 | 50 | 3.72 | 16 | 946.3 |

The insulin was weighed into a beaker, the HCl and the water added and the mixture stirred until all the insulin had dissolved. The NaOH was then added causing the insulin to precipitate and then re-dissolve to form a clear solution. The trehalose was added to the neutral solution. The quantities of materials used is shown in Table 1.

Spray Drying

Spray drying was carried out using a Mini spray drier and a Schlick two fluid nozzle. The atomisation pressure (Compressed air) was 4 bar (30 l/min) inlet temperature 130° C. to maintain an outlet of ˜75° C. A pre-weighed collection pot was fitted to the bottom of the filter housing to collect the powder.

The flow properties of RDD/03/028 [ex. Jetpharma bag filter system] and RDD/03/030 [standard cyclone collection] as exhibited by filling variance were assessed using a custom made dosator filling apparatus. The target fill weight was 4.5 mg. The dramatic reduction in filling variance (Table 2) is indicative of the improved flow of powders produced according to the invention.

TABLE 2

| | | | | | Fine | | |
|---|---|---|---|---|---|---|---|
| | | | | | particle | | |
| | | | | | dose | | |
| | | | Emitted | | <3.3 μm | Mean | |
| Batch | | Collection | dose | MMAD | (of | Fill | |
| Number | Formulation | type | (%) | microns | detected) | weight | Variance |
| RDD/03/030 | 20:80% w/w | Mini SD | 82.8 | 2.49 | 57.8 | 4.78 | 0.090 |
| | insulin:trehalose | Cyclone | | | | | |
| RDD/03/028 | 20:80% w/w | Mini SD | 79 | 2.57 | 44.8 | 4.54 | 0.036* |
| | insulin:trehalose | Jetpharma | | | | | |

*denotes statistically significant difference (p < 0.05)

### EXAMPLE 2

The following experiment set out to replicate the results achieved in Example 1, but utilised pure insulin (no carbohydrate). The collection of microparticles from cyclone was included as a control. Two different filters were also tested; a metal filter and a polytetrafluoroethylene filter.

Solution Preparation

Two pure insulin batches were made using the Mobile Minor spray dryer; a batch of insulin (RDD/04/001) was prepared using the bag filter collection system, a second batch was prepared using the metal filter collection system. (RDD/04/002). Diosynth insulin (EM/03/096) was used to prepare both batches. The formulation details of each batch are shown in Table 3. In each case a 5% w/v total solids solution was obtained.

TABLE 3
Preparation of Solutions for Spray Drying

| | | 0.1M HCL | 1.0M NaOH | |
|---|---|---|---|---|
| Batch | Insulin (g) | (ml) | (ml) | Water (ml) |
| RDD/04/001 | 100 g | 62.5 ml | 93 ml | 1844.5 ml |
| RDD/04/002 | 100 g | 62.5 ml | 93 ml | 1844.5 ml |

The insulin was weighed into a 3 litre beaker, the HCl and 1 litre of water added and the mixture stirred until all the insulin had dissolved. The NaOH and the remaining water was then added causing the insulin to precipitate and then re-dissolve to form a clear solution.

Spray Drying

Spray drying was carried out using a Niro Mobile Minor spray drier and a Schlick two fluid nozzle. The atomisation pressure (Compressed air) was 7 bar (131/sec), inlet temperature 220° C. to maintain an outlet of ˜75° C., drying air 16-18 mm.wg. The bag filter system had a filter surface area of 1.9 m². It 10 was insulated with foam rubber. Reverse jetting was done to each of the three bags in turn every 5 minutes at 4 bar. A pre-weighed glass collection jar was fitted at the bottom of the filter housing to collect the powder.

Insulin Analysis

HPLC and SEC analysis of the powders was carried The levels of desamido insulin (A21), insulin related substances (IRS) and high molecular weight proteins (HMWP) were similar to those found in the starting material, insulin. This shows that the insulin was not degraded by the formulation or spray drying processes.

Scanning Electron Microscopy

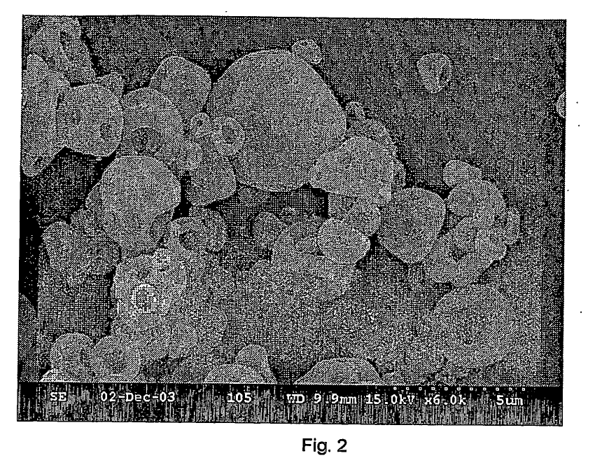

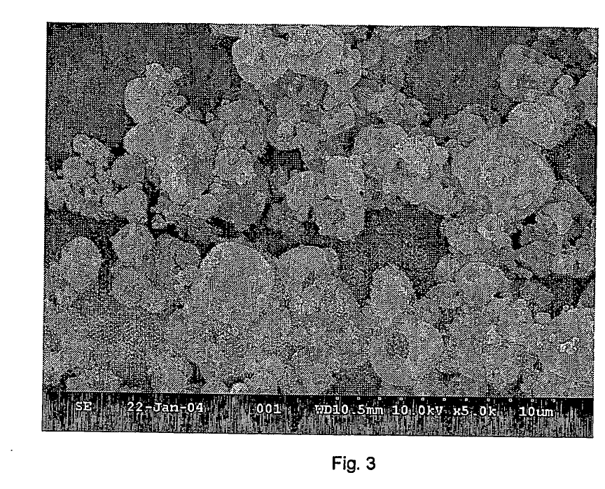

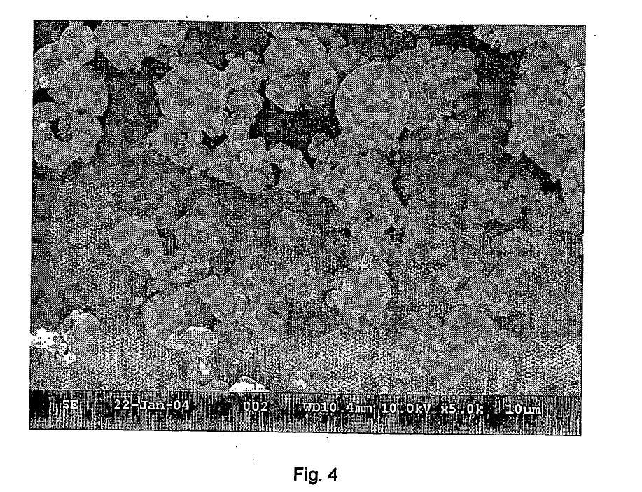

SEM showed that the particles produced for both formulations were very similar—highly convoluted, collapsed spheres. As with all filter collected batches, there were a significantly greater number of sub-micron particles in the formulation (FIGS. 3 & 4). SEM also showed that cyclone collected particles were predominantly of one size population (FIG. 2)

Density

Bulk and tap density were measured by standard methods. The Carr's index—a measure of flowability—can be calculated using the formula
C.I.=((tap−bulk)/tap)×100
and is expressed as a percentage. The results are shown in Table 4.

The values obtained for these batches suggest that the microparticles should have reasonable flow properties. A comparison of the particle size distributions of powders produced using the filters and cyclone collection systems is also shown in Table 4

TABLE 4

| Batch | | | Mean | % | % | % | % | % | % |
|---|---|---|---|---|---|---|---|---|---|
| Number | Formulation | Separator | (Fm) | <0.54 μm | <0.74 μm | <1.0 μm | <1.4 μm | <2.5 μm | <3.4 μm |
| RDD/03/008 | 100% | Cyclone | 1.2 | 0.66 | 7.2 | 29.4 | 68.8 | 98.4 | 99.9 |
| | insulin | (MSD) | | | | | | | |
| RDD/03/027 | 100% | Jetpharma | 1.1 | 0.86 | 10.5 | 69.8 | 79.5 | 99.2 | 99.8 |
| | insulin | (MSD) | | | | | | | |
| RDD/03/105 | 100% | Cyclone | 1.2 | 0.46 | 6.6 | 30.5 | 73.3 | 99.8 | 100.0 |
| | insulin | (MM) | | | | | | | |
| RDD/04/001 | 100% | Bag filter | 0.92 | 5.6 | 31.6 | 62.5 | 87.1 | 99.1 | 99.9 |
| | insulin | (MM) | | | | | | | |
| RDD/04/002 | 100% | Metal filter | 0.91 | 5.3 | 31.6 | 62.4 | 87.0 | 99.2 | 99.9 |
| | insulin | (MM) | | | | | | | |

Using an Amherst Aerosizer Aerodisperser to determine the aerodynamic diameter (Time of Flight measuring principle), particle size distributions were obtained for each formulation. Each sample was measured in triplicate. By expressing the distribution by number, the % of particles below a size threshold can be obtained from the measurement.

TABLE 5
yields, flow properties and device performance

| | | | | | | FPD |
|---|---|---|---|---|---|---|
| Batch | | | CCI | Yield | Emitted | <3.3 |
| Number | Formulation | Separator | (&) | (%) | Dose | μm |
| RDD/03/008 | 100% insulin | Cyclone | 41 | NT | 83 | 52.7 |
| | | (MSD) | | | | |
| RDD/03/027 | 100% insulin | Jetpharma | 23 | NT | 82 | 52.4 |
| | | (MSD) | | | | |
| RDD/03/105 | 100% insulin | Cyclone | 45 | 69 | 90.8 | 52% |
| | | (MM) | | | | |
| RDD/04/001 | 100% insulin | Bag filter | 25 | 80 | 91.5 | 49% |
| | | (MM) | | | | |
| RDD/04/002 | 100% insulin | Metal filter | 24 | 75 | 91.7 | 49% |
| | | (MM) | | | | |

The filter collection system results in a much higher recovered yield of is material—up to 80%—when compared to the 60% yield seen with cyclone collection. The larger the batch size the better the recovery is as losses to the filter surfaces are constant.

The bag and metal filters show enormous potential as a method of collecting spray-dried particles with a recovered yield of greater than 90%.

In summary, inhalable fine powders with increased yields of fine particles are collected successfully using a filter bag assembly in series with a conventional spray drier. These powders are composed of a critical ratio of inhalable microparticles (IMP) that target deep lung delivery, as well as smaller nano binding particles (NBP) adsorbed on their surface to aid the aggregation and flow of the IMPs. This is a surprising result. The powders have unique flow properties that aid in their filling into blisters. Interestingly, their dispersion is not affected.

1. A composition comprising microparticles, the microparticles comprising a bioactive agent wherein,
(1) at least 40% of the microparticles are from 1 μm to 5 μm aerodynamic diameter;
(2) at least 35% of the microparticles are less than 1 μm; and
(3) at least 2% of the microparticles are less than 0.5 μm.

2. The composition according to claim 1, wherein,
(4) at least 40% of the microparticles are from 1 μm to 5 μm;
(5) at least 50% of the microparticles are less than 1 μm; and
(6) at least 5% of the microparticles are less than 0.5. μm.

3. The composition according to claim 1, wherein at least 20% of the microparticles are less than 0.75 μm.

4. The composition according to claim 1, wherein all the microparticles in the composition are less than 4 μm aerodynamic diameter.

5. The composition according to claim 1, wherein the microparticles further comprise a carbohydrate matrix, within which the bioactive agent is dispersed.

6. The composition according to claim 5, wherein the carbohydrate is trehalose.

7. The composition according to claim 5, wherein the bioactive agent is a protein or peptide.

8. The composition according to claim 1, wherein the bioactive agent is insulin.

9. The composition according to claim 1, wherein the microparticles are reversibly agglomerated.

10. A method for the preparation of a composition comprising microparticles, the microparticles comprising a bioactive agent wherein,
(1) at least 40% of the microparticles are from 1 μm to 5 μm aerodynamic diameter,
(2) at least 35% of the microparticles are less than 1 μm; and
(3) at least 2% of the microparticles are less than 0.5 μm; wherein said method comprises spray drying a solution or suspension of the agent in a spray dry apparatus under conditions suitable to provide microparticles of less than 5 μm diameter, the apparatus having a separator for separating the spray dried microparticles from a gas stream, and collecting the microparticles.

11. The method according to claim 10, wherein the separator is a metal, polyester or polytetrafluoroethylene filter.

12. The method according to claim 10, wherein the spray dryer comprises a two fluid nozzle.

13. The method according to claim 10, wherein spray drying is carried out at an outlet temperature of at least 70° C.

14. The method according to claim 10, wherein spray drying is carried out with an atomization pressure of at least 6 bar and an airflow of at least 10 l/second.

15. The method according to claim 10, wherein the yield of the bioactive agent in the microparticles is at least 70% of that present in the solution or suspension.

16. The method according to claim 10, wherein the spray dried product has an emitted dose of at least 60%.

17. A particulate composition comprising a bioactive agent, obtainable by spray drying a solution or suspension of the agent according to a method for the preparation of a composition comprising microparticles, the microparticles comprising a bioactive agent wherein,
(1) at least 40% of the microparticles are from 1 μm to 5 μm aerodynamic diameter;
(2) at least 35% of the microparticles are less than 1 μm; and
(3) at least 2% of the microparticles are less than 0.5 μm;
wherein said method comprises spray drying a solution or suspension of the agent in a spray dry apparatus under conditions suitable to provide microparticles of less than 5 μm diameter, the apparatus having a separator for separating the spray dried microparticles from a gas stream, and collecting the microparticles.

18. A device for the pulmonary delivery of a bioactive agent, the device comprising a composition comprising microparticles, the microparticles comprising a bioactive agent wherein,
(1) at least 40% of the microparticles are from 1 μm to 5 μm aerodynamic diameter;
(2) at least 35% of the microparticles are less than 1 μm; and
(3) at least 2% of the microparticles are less than 0.5 μm.

19. The device according to claim 18, wherein the fine particle dose is more than 30% of detected bioactive is less than 3.3 μm.
